(19) United States
(12) Patent Application Publication
(10) Pub. No.: US 2026/0035329 A1
(43) Pub. Date: Feb. 5, 2026

(54) PROCESS FOR PREPARING AN AQUEOUS LACTATE SOLUTION

(71) Applicant: PURAC Biochem B.V., Gorinchem (NL)

(72) Inventors: Fesia Lestari LAKSMANA, Gorinchem (NL); Anne FOKKEMA, Gorinchem (NL)

(21) Appl. No.: 19/356,514

(22) Filed: Oct. 13, 2025

Related U.S. Application Data
(63) Continuation of application No. PCT/EP2024/059472, filed on Apr. 8, 2024.

(30) Foreign Application Priority Data
Apr. 14, 2023 (EP) 23168042.2

(51) Int. Cl. C07C 51/02; C07C 51/41; C07C 59/08

(52) U.S. Cl. CPC C07C 51/02; C07C 51/412; C07C 59/08

(57) ABSTRACT

The present invention provides a process for manufacturing a lactate solution which results in an economically attractive process of producing lactic acid when the lactate solution is subjected to water-splitting electrodialysis, solid-state magnesium lactate or an aqueous magnesium lactate suspension is reacted with KOH to form a first suspension of solid magnesium hydroxide in a first potassium lactate solution comprising magnesium ions, the amount of excess OH ions in the first suspension being in the range of 20-150 ppm. After solid-liquid separation, the first potassium lactate solution is subjected to a further reaction with KOH to form a second suspension of solid magnesium hydroxide in a second potassium lactate 10 solution, the amount of excess OH ions in the second suspension being above 300 ppm. The second suspension is separated, and the second potassium lactate solution has a magnesium ion content of less than 50 ppm.

### CROSS-REFERENCE TO RELATED APPLICATIONS

The present application is a continuation of International Application No. PCT/EP2024/059472, filed Apr. 8, 2024, which claims priority to European Patent Application No. 23168042.2, filed Apr. 14, 2023, all of which are hereby incorporated by reference herein in their entireties.

### TECHNICAL FIELD OF THE INVENTION

The present invention relates to a process for preparing an aqueous lactate solution from a magnesium lactate comprising medium. The invention also relates to further processing of such a solution to generate lactic acid.

### BACKGROUND OF THE INVENTION

Lactic acid (LA), also known as 2-hydroxypropanoic acid, has many commercial applications, including use in the food industry and as monomer for the manufacture of biodegradable and/or renewable polymers.

Most of the commercial processes for the preparation of lactic acid are based on the fermentation of carbohydrates by micro-organisms. These processes require strict control of temperature and pH. A common feature to all the fermentation processes is the need to neutralize the acids excreted by the micro-organisms in the process. A drop in pH below a critical value, depending on the micro-organism used in the process, could damage the micro-organism's metabolic process and bring the fermentation process to a stop. Therefore, fermentation processes often result in the formation of lactic acid in the form of a salt, namely, the salt of the base added to keep the pH of the fermentation medium in the pH range acceptable to the microorganism.

Despite the many efforts in the field, it remains a challenge to generate lactic acid in high purity from the aqueous medium comprising the lactate salt, with the challenge not only residing in obtaining the high purity, but also in obtaining it in a manner which can be operated reliably and consistently in an energy-efficient manner. The latter is required, among other features, to make lactic acid an attractive monomer for polymer manufacture.

An example of a process for the preparation of lactic acid is described in WO98/22611. This patent publication describes a process for producing lactic acid through the steps of producing lactic acid through fermentation, adding alkaline earth base such as calcium base to form alkaline earth lactate such as calcium lactate, removing biomass, reacting the alkaline earth base with an ammonium source to form ammonium lactate, and recovering lactic acid therefrom by salt splitting electrodialysis.

WO2005/123647 describes a process for the production of lactic acid from a magnesium lactate-comprising medium which may be provided via fermentation. The magnesium lactate is reacted with a hydroxide of sodium, potassium, calcium and/or ammonium to form magnesium hydroxide and the corresponding monovalent and/or divalent lactate salt. As means to convert the lactate salt into lactic acid the document suggests bipolar electrodialysis or the addition of a strong mineral acid. However, such conversions are not included in the processes exemplified in this document.

WO2011/095631 describes a process for the preparation of lactic acid comprising the steps of: a) providing an aqueous medium comprising magnesium lactate; b) adding to the aqueous medium comprising magnesium lactate a monovalent base to form an aqueous medium comprising a water soluble monovalent lactate salt and a solid magnesium base; c) separating the magnesium base from the aqueous medium comprising the water soluble monovalent lactate salt; d) adjusting the concentration of the monovalent lactate salt in the aqueous medium to a value between 10 and 30 wt. %, e) subjecting the aqueous medium comprising the monovalent lactate salt to water-splitting electrodialysis, to produce a first solution comprising monovalent base and a second solution comprising lactic acid and monovalent lactate salt, the electrodialysis being carried out to a partial conversion of 40 to 98 mole %; f) separating the second solution comprising lactic acid and monovalent lactate salt into lactic acid and a solution comprising the monovalent lactate salt by vapour-liquid separation; g) recycling the solution of step f) comprising the monovalent lactate salt to step d). It has been found that while water-splitting electrodialysis in principle is an attractive method to generate high purity lactic acid, it is not easy to carry it out in a manner which is economically attractive due to its sensitivity and its inherent energy requirements. This is the more so if the starting material is a lactate solution derived from a fermentation process, which is associated with the possible presence of a wide variety of possible organic and inorganic contaminants which may interfere with the water-splitting electrodialysis, such as divalent cations.

There is a need in the art for a process for manufacturing a lactate solution which results in an economically attractive process of producing lactic acid when the lactate solution is subjected to water-splitting electrodialysis. The present invention provides such a process.

### SUMMARY OF THE INVENTION

The invention pertains to a process for preparing an aqueous potassium lactate solution with a magnesium ion content of less than 50 ppm, the process comprising the steps of
in a first reaction step reacting solid-state magnesium lactate in an aqueous magnesium lactate suspension with KOH to form a first suspension of solid magnesium hydroxide in a first potassium lactate solution comprising magnesium ions, the reaction taking place under such conditions that the amount of excess OH ions in the suspension is in the range of 20-300 ppm,
subjecting the first suspension to a solid-liquid separation step to separate solid magnesium hydroxide from the first potassium lactate solution,
in a second reaction step subjecting the first potassium lactate solution resulting from the solid-liquid separation step to a further reaction with KOH to form a second suspension of solid magnesium hydroxide in a second potassium lactate solution, the reaction taking place under such conditions that the amount of excess OH ions in the suspension is above 300 ppm,
subjecting the second suspension to a solid-liquid separation step to separate solid magnesium hydroxide from the second potassium lactate solution, wherein the second potassium lactate solution has a magnesium ion content of less than 50 ppm.

The present invention relies on a number of key features in combination.

A first key feature of the process according to the invention is that it is aimed at the manufacture of a potassium lactate solution rather than a sodium lactate solution. It has been found that potassium lactate solutions are more attractive for processing in water-splitting electrodialysis than sodium lactate solutions. In chemistry, sodium salts are the generally preferred alkalimetal salts, for reasons of cost, safety, and availability.

A further key feature of the process resides in an excess OH ions in the first reaction step of 20-150 ppm and an excess OH ions in the second reaction step of more than 300 ppm, and combining the two reaction steps with an intermediate product separation. These features in combination have been found to result in a final KOH solution with a very low content of divalent alkaline earth metal components, in particular magnesium and calcium, while at the same time ensuring that magnesium hydroxide can be recovered form the process in an efficient and cost-effective manner.

The process according to the invention can in particular be used for the manufacturing of lactic acid. Therefore, the invention also relates to a use of the aqueous potassium lactate solution with a magnesium content of less than 50 ppm obtained in accordance with any one of the preceding claims in the manufacture of lactic acid.

Finally, the invention relates to a process for manufacturing lactic acid, comprising the steps of:
preparing an aqueous potassium lactate solution with a magnesium content of less than 50 ppm according to the invention,
providing the aqueous potassium lactate solution with a magnesium content of less than 50 ppm to a reaction step in which potassium lactate is converted to lactic acid, e.g. by reacting the potassium lactate with a strong inorganic acid or through electrodialysis.

The invention will be discussed in more detail below.

### DETAILED DESCRIPTION OF THE INVENTION

The starting material in the process according to the invention is magnesium lactate in the solid state. The magnesium lactate may be provided in the form of a slurry, but provision in the form of a solid, e.g., in the form of a filter cake or in the form of dried crystals is also envisaged. Magnesium lactate in the solid state may be suspended in water where part of the magnesium lactate will be dissolved in water and part will remain as solid. Alternatively, magnesium lactate in the solid state may also be suspended in potassium lactate solution derived from the first or second filtration.

Both forms (either magnesium lactate in the solid state or magnesium lactate as aqueous suspension) will hereafter be referred to as “magnesium lactate”.

The magnesium lactate is combined with KOH to form a reaction medium. For ease of processing, the KOH will generally be provided in the form of a solution. The concentration of the KOH solution will depend on the way in which the magnesium lactate is provided. If the magnesium lactate is provided in the form of a filter cake or dried crystals, a more dilute KOH solution may be attractive. On the other hand, if the magnesium lactate is provided in the form of a relatively dilute slurry, a highly concentrated KOH solution will desirably be used. Alternatively, KOH may be provided in the solid form such as pellets. Reaction of the magnesium lactate with KOH then results in the first suspension of solid magnesium hydroxide in a first potassium lactate solution.

In one embodiment, the aqueous magnesium lactate suspension has a magnesium lactate content (calculated as magnesium lactate anhydrate) of at least 15 wt. %. Preferably, the magnesium lactate content is at least 20 wt. %, and in particular at least 25 wt. %.

Preferably, the aqueous magnesium lactate suspension has a magnesium lactate content (calculated as magnesium lactate anhydrate) of at most 50 wt. % of the total weight of the suspension.

In one embodiment, the solid-state magnesium lactate comprises at least comprises at least 60 wt. %, preferably at least 70 wt. %, more preferably at least 80 wt. % and in particular at least 85 wt. % magnesium lactate (calculated as magnesium lactate anhydrate) of the total weight of the solid.

Preferably the first reaction step is performed under mixing. In this way, high local hydroxide concentrations in the suspension can be avoided.

In the first reaction step, the magnesium lactate is reacted with KOH to form a first suspension of solid magnesium hydroxide in a first potassium lactate solution. The amount of KOH provided is such that the amount of excess OH ions in the first suspension is in the range of 20-150 ppm. It has been found that selecting an amount of excess OH ions in the stipulated range during this reaction step ensures that crystals with good filtration properties are obtained. Such crystals can easily be removed from the potassium lactate solution without e.g. using an excessive size of separation equipment and/or using a large amount of water to wash the potassium lactate from Mg (OH) 2 crystals

It is preferred that the amount of excess OH ions in the first suspension is at most 125 ppm, more preferably at most 100 ppm, in some embodiments at most 70 ppm. Additionally, or in combination, it may be preferred for the excess of OH ions to be at least 25 ppm, in particular at least 30 ppm and more particularly at least 35 ppm.

The excess of OH ions in the solution is determined by potentiometric titration as follows: Liquid part of the suspension is taken by using a syringe equipped with a 0.45 um filter to remove any residual solids in the sample. 5-10 g of the filtered samples is weighed, accurately to 0.0001 g, into a titration vessel. 70 mL milli-Q water is added to the sample. The sample is titrated with 0.01 M hydrochloric acid and the difference of the electrical potential is measured at different volumes of hydrochloric acid addition. The first equivalence point is determined by finding the maximum slope of the titration curve. Hydroxide concentration in the sample is then calculated using the following formula:

$$OH [ ppm ] = \frac{\begin{matrix}HCl volume at 1 st equivalence point [ mL ] \times \\ 0.01 [ \frac{mol}{L} ] \times 17.007 [ \frac{g}{mol} ] \times {10}^{3}\end{matrix}}{Sample weight}$$

It is preferred for the vast majority of the magnesium present in the system to be recovered from the process after the first reaction step. In particular, it is preferred that of the total amount of magnesium lactate in the starting aqueous magnesium lactate suspension at least 75 wt. % is converted into potassium lactate and magnesium hydroxide in the first reaction step, in particular at least 85 wt. %, more in particular at least 90 wt. %, still more in particular at least 95 wt. %, even more in particular at least 98 wt. %. Such conversion can be achieved, by controlling the pH and achieving the excess of OH ion concentration as described in the current embodiments. The reaction time is typically between 30 to 60 min and at least 10 min.

The conversion can be calculated from the amount of magnesium lactate which is present in the recovered solid magnesium hydroxide. This value can be determined based on the amount of magnesium lactate provided in the first reaction step and the amount of residual magnesium lactate present in the Mg(OH)₂ filter cake recovered after separating the solid magnesium hydroxide from the first potassium lactate solution.

The suspension of magnesium hydroxide in potassium lactate solution is subjected to a solid-liquid separation step to separate solid magnesium hydroxide from the first potassium lactate solution. The solid liquid separation step can be carried out in manners known in the art. As the magnesium hydroxide product has good filtration properties, a conventional filtration using vacuum, high pressure as well as centrifugal forces such as a horizontal belt filter, pressurized belt filter or peeler centrifuge will be suitable to separate the solid magnesium hydroxide from the first potassium lactate solution.

The amount of excess OH ions in the range of 20-150 ppm in the first suspension is suitable to provide crystals with good filtration properties, but will also result in a first potassium lactate solution containing residual magnesium ions. In order to make the lactate solution better suited for electrodialysis, the first potassium lactate solution is subjected to a further reaction with KOH to form a second suspension of solid magnesium hydroxide in a second potassium lactate solution. It was found that when the amount of excess OH ions in the second suspension is above 300 ppm, a second potassium lactate solution with a magnesium ion content of less than 50 ppm is obtained. The second potassium lactate solution is separated from the solid magnesium hydroxide by a solid-liquid separation step, thus providing a lactate solution which is better suited for electrodialysis, resulting in a process of producing lactic acid with lower downtime and thus an economically more attractive process of producing lactic acid.

It is preferred that in the second reaction step the excess OH ions in the second suspension is above 400 ppm, in particular above 500 ppm, more in particular above 650 ppm, and generally above 800 ppm. In this way, a second potassium lactate solution with a lower magnesium ion content is obtained. Preferably, the excess OH ions in the second suspension is in the second reaction step at most 10000 ppm, since magnesium hydroxide crystals obtained at relatively high levels of excess OH ions have a lower filterability and are therefore more difficult to remove from a suspension by filtration. Using excess OH ions above this concentration, the magnesium ion content in the second potassium solution would hardly reduce further.

In an embodiment, the difference between the excess OH ions in the first suspension in the first reaction step and the excess OH ions in the second suspension in the second reaction step is at least 100 ppm, in particular at least 200 ppm, more in particular at least 300 ppm, still more in particular at least 400 ppm, and at most 1200 ppm, in particular at most 1000 ppm, more in particular at most 850 ppm, still more in particular at most 700 ppm, even more in particular at most 10000 ppm. In this way, the higher excess of OH ions in the second suspension in the second reaction step as compared to the suspension in the first reaction step leads to a stronger reduction of the magnesium ion content of the first potassium lactate solution. Thus, a second potassium lactate solution having a magnesium ion content of less than 50 ppm is obtained.

The concentration of OH ions in the first suspension in the first reaction step and the second suspension in the second reaction step depends on both the temperature and the pH of the respective suspension. At a higher temperature, the ionisation of water increases, thus increasing the concentration of OH ions and increasing the pH. On the other hand, the addition of OH ions to the suspension in the form of a base also increases the concentration of OH ions and the pH. Thus, the higher excess OH ions in the second suspension as compared to the first suspension can be achieved by carrying out the second reaction step at a temperature which is higher than the temperature at which the first reaction step is carried out, by carrying out the second reaction step at a pH which is higher than the pH of the first reaction step, or by carrying out the second reaction step at a temperature and pH which are both higher than the temperature and pH of the first reaction step.

To achieve a difference between the excess OH ions in the first reaction step and the excess OH ions in the second reaction step of at least 100 ppm, it is preferred that the second reaction step is carried out at a temperature which is at least 20° C. higher than the temperature at which the first reaction step is carried out, and/or that the second reaction step is carried out at a pH which is at least 0.5 higher than the pH at which the first reaction step is carried out.

In an embodiment, the second reaction step is carried out at a temperature which is at least 25° C. higher than the temperature at which the first reaction step is carried out, and at most 75° C. higher. In this way, the excess OH ions in the second reaction is increased as compared to the first reaction step, and a second potassium lactate solution with a magnesium ion content of less than 50 ppm is obtained.

Preferably, the first and the second reaction step are carried out at a temperature in the range of 20-100° C., in particular 25-90° C., more in particular 30-80° C., the temperature in the two steps being selected individually. Preferably, the first reaction step is carried out at a temperature in the range of 20-70° C. and the second reaction step is carried out at a temperature in the range of 40-90° C. If the temperatures of the first and the second reaction step are chosen such that the difference in excess OH ions between the first and the second reaction step is less than a desired value, the difference in excess OH ions may be increased by the addition of a base in the second reaction step.

In an embodiment, the pH in the second reaction step is at least 0.7 above the pH in the first reaction step, in particular at least 0.8. The pH in the second reaction step generally is at most 2.5 above the pH in the first reaction step, in particular at most 2. Preferably, the first and the second reaction step are carried out to obtain a pH in the step in the range of 9.5-14, in particular in the range of 10-12.5, the pH in the two steps being selected individually. If the pH of the first and the second reaction step are chosen such that the difference in excess OH ions between the first and the second reaction step is less than a desired value, the difference in excess OH ions may be increased by performing the second reaction step at a higher temperature than the first reaction step.

Preferably, the second reaction step is carried out at temperature which is at least 20° C. higher than the temperature at which the first reaction step is carried out, and the pH of the second reaction step is less than 0.5 above the pH of the first reaction step. Alternatively, the pH in the second reaction step is at least 0.5 above the pH in the first reaction step and the second reaction step is carried out at a temperature which is at most 20° C. higher than the temperature at which the first reaction step is carried out. In this way, it is not necessary to substantially increase both the pH and the temperature in the second reaction step as compared to the first reaction step to increase the excess OH ions in the first reaction step as compared to the second reaction step. Since only one of the temperature and the pH is modified substantially between the first and the second reaction step, this results in a more economically attractive way of achieving the increased excess OH ions in the second reaction step as compared to the first reaction step.

It is preferred that the second potassium lactate solution has a magnesium ion content of less than 25 ppm, in particular less than 15 ppm, more in particular less than 10 ppm, still more in particular less than 5 ppm, or even less than 2 ppm. In this way, the lactate solution is even better suited for a subsequent step of electrodialysis in the manufacturing of lactic acid.

Preferably, the process for preparing an aqueous potassium lactate solution having a magnesium ion content of less than 50 ppm is carried out in a continuous manner.

An aqueous potassium lactate solution with a magnesium content of less than 50 ppm is especially useful in the manufacture of lactic acid. Therefore, the invention relates in further aspects to the use of the aqueous potassium lactate solution with a magnesium content of less than 50 ppm according to the invention as a starting material in the manufacture of lactic acid, and to a process for manufacturing lactic acid.

Water splitting electrodialysis is a common way of converting potassium lactate in lactic acid and potassium hydroxide. Water splitting electrodialysis in particular allows the direct conversion of a lactate salt into lactic acid and base. In this type of electrodialysis bipolar membranes are generally used to split water into H⁺ and OH⁻, respectively, which combine with the anion and cation of the lactate salt, respectively, resulting in the production of separate solutions of lactic acid and base. However, the presence of divalent cations such as magnesium in the dialysis process can result in fouling of the membranes and decreasing power efficiency. Thus, the electrodialysis equipment needs to be cleaned at a regular basis, resulting the interruption of the electrodialysis and decreasing the efficiency of lactic acid production. The aqueous potassium lactate solution with a magnesium content of less than 50 ppm according to the invention is less prone to affect the electrodialysis equipment. Thus, less maintenance of the equipment and therefore less interruption of the electrodialysis is required. Lactic acid and potassium hydroxide can therefore be manufactured in an economically more attractive way.

In an embodiment, a process for manufacturing lactic acid comprises the steps of
preparing an aqueous potassium lactate solution with a magnesium content of less than 50 ppm according to the invention,
providing the aqueous potassium lactate solution with a magnesium content of less than 50 ppm to a reaction step in which potassium lactate is converted to lactic acid and potassium hydroxide through electrodialysis.

As an alternative for water splitting electrodialysis, the potassium lactate may be converted to lactic acid by reacting the potassium lactate with a strong inorganic acid.

The potassium hydroxide produced by electrodialysis of potassium lactate is preferably recycled to the first reaction step and/or the second reaction step for reaction with magnesium lactate. In this way, a more cost effective process is achieved. Since the potassium hydroxide produced by the electrodialysis generally has a temperature of about 40° C., the first and second reaction step are preferably performed at at least 40° C., in order to provide an even more cost effective process.

Lactic acid is commonly produced by fermenting a carbohydrate source to lactic acid in the presence of a microorganism. In order to neutralise the fermentation medium, a base is added to the medium as a neutralising agent, resulting in the formation of an aqueous fermentation medium comprising lactate. Thus, the process for the manufacture of lactic acid preferably comprises the step of fermenting a carbohydrate source to lactic acid in the presence of a microorganism, with the addition of magnesium hydroxide as neutralising agent, to form an aqueous fermentation medium comprising magnesium lactate. This aqueous medium can then serve as a basis for the preparation of the aqueous potassium lactate solution according to the invention. Optionally, the aqueous medium is subjected to a biomass removal step prior to the preparation of the aqueous potassium lactate solution, in order to substantially remove the microorganism used in the fermentation process. Optionally, the aqueous medium is concentrated prior to the preparation of the aqueous potassium lactate solution, to provide a magnesium lactate slurry which can be fed to the first reaction step. Optionally, a liquid-solid separation step is performed to obtain solid magnesium lactate. Optionally, the magnesium lactate dissolved in the aqueous fermentation medium is crystallised to obtain solid magnesium lactate. In this way, the magnesium lactate yield of the fermentation is increased. Solid magnesium lactate can then be used to prepare the aqueous magnesium lactate suspension

Preferably, magnesium hydroxide recovered from the first and/or second suspension as obtained from subjecting the first and/or second potassium lactate suspension to solid-liquid separation to the fermentation step as a neutralising agent. This results in a more cost effective process of producing lactic acid.

Preferably the magnesium lactate slurry has a magnesium lactate content (calculated as magnesium lactate anhydrate) of at least 30 wt. % based on the total weight of the slurry. Such a magnesium lactate concentration is advantageous for preparing a potassium lactate solution with a relatively high concentration of potassium lactate, which in turn can be used to produce a relatively high amount of lactic acid. Said magnesium lactate content can for example be achieved by heating the aqueous medium to remove water and concentrate the aqueous medium.

The following example will illustrate the practice of the present invention in some of the preferred embodiments. Other embodiments within the scope of the claims will be apparent to one skilled in the art.

### Examples

The examples 1-2 show the effect of either relatively low or relatively high amounts of excess OH ions in both the first and second reaction step on the filterability of magnesium hydroxide (Mg(OH)₂ particles, and on the moisture content, potassium (K) content and magnesium (Mg) lactate content of the Mg (OH) 2 cake obtained after the first reaction step. Furthermore, the examples 3-5 show the effect of relatively low amounts of excess OH ions in the first step and relatively high amounts of excess OH ions in the second step on the magnesium ion content of the second potassium lactate (KL) solution.

In a first reaction step, 42-47 wt. % magnesium lactate (as anhydrate) slurry was reacted continuously for 1 hour in a first reactor with 11-12 wt. % KOH solution at 50° C. and at the pH indicated in table 1 below, resulting in the formation of a first suspension of solid magnesium hydroxide (MgOH₂) and a first potassium lactate solution. After one hour of residence time in the first reactor, Mg(OH)₂ was separated from the first potassium lactate solution by filtration through 7 μm mesh size.

The first potassium lactate solution was then sent to a second reactor and in a second reaction step reacted continuously for 0.5 hour with 11-12 wt. % KOH solution at either a higher temperature than the first reaction step or at a higher pH than the first reaction step, as indicated in table 1 below. The second reaction step resulted in the formation of a second suspension of solid Mg(OH)₂ and a second potassium lactate solution. After 0.5 hour of residence time in the second reactor, Mg(OH)₂ was separated from the first potassium lactate solution by filtration through a filter cloth with 1 μm mesh size.

After the first reaction step, the filterability of the Mg(OH)₂ particles and the moisture content, potassium content and magnesium lactate content of the Mg(OH)₂ cake were determined as shown in table 2.

After the second reaction step, the magnesium ion content of the second potassium lactate solution was determined as shown in table 3.

TABLE 1

| | Reactor 1 | | | Reactor 2 | | |
|---|---|---|---|---|---|---|
| | | | [OH] | | | [OH] |
| Example | T ° C. | pH | ppm | T ° C. | pH | ppm |
| 1 | 50 | 11.4 | 322 | 50 | 12 | 675 |
| 2 | 50 | 10.8 | 38 | 50 | 10.8 | 50 |
| 3 | 50 | 10.8 | 54 | 50 | 12 | 666 |
| 4 | 50 | 11.1 | 65 | 50 | 12 | 675 |
| 5 | 50 | 11.1 | 65 | 80 | 11.2 | 721 |

### Results

The results show that when the amount of excess OH ions in the first reactor was relatively high, such as in example 1, the filterability of the Mg(OH)₂ formed in the first reaction step exhibited a filterability of 109 kg dry solids/m²h, indicating it was relatively difficult to separate the Mg(OH)₂ formed under these conditions from the potassium lactate solutions.

When the amount of excess OH ions in the first and in the second reactor was <100 ppm such as shown in example 2, the filterability of the Mg(OH)₂ formed in the first reaction step exhibited a filterability of above 150 kg dry solids/m²h while the second potassium lactate solution contains more than 50 ppm Mg.

However, when the amount of excess OH ions in the first reactor was less than 150 ppm in the first reactor, as in examples 3-5, the Mg(OH)₂ formed in the first reaction step has a filterability of >150 kg dry solids/m²h, indicating the Mg(OH)₂ formed in the first reaction step can be separated from the potassium lactate solutions relatively easily. Since the separation of Mg(OH)₂ from the first potassium lactate solution is required before sending the first potassium lactate solution to the second reactor for manufacturing the second potassium lactate solution with less than 50 ppm magnesium ions, a relatively easy separation of Mg(OH)₂ from the first potassium lactate solution after the first reactor contributes to a more efficient production of the second potassium lactate solution.

Additionally, the Mg(OH)₂ cake under the conditions of experiments 3-5 contains a relatively low amount of potassium, making the Mg(OH)₂ obtained after the second reaction step suitable to supply as neutralising agent to a fermentation step.

TABLE 2

| | | | Residual component | | |
|---|---|---|---|---|---|
| | | | in Mg(OH)₂ cake | | |
| | | | | | (Mg) |
| | Filterability | Mg in KL | Moisture | K | Lactate |
| Example | (kg d.s./m²h) | (ppm) | (% w) | (ppm) | (% w) |
| 1 | 109 | 57 | 73.5 | 2015 | 0.7 |
| 2 | 188 | 169 | 71.3 | 436 | 1.7 |
| 3 | 169 | 138 | 75.9 | 338 | 1.2 |
| 4 | 314 | 145 | 71.2 | 786 | 1.0 |
| 5 | 314 | 145 | 71.2 | 786 | 1.0 |

Furthermore, as can be seen in table 3 below, the second potassium lactate solutions obtained in the processes under the conditions of examples 3-5 contained less than 50 ppm magnesium ion. In fact, when the first reaction step took place at an excess amount of OH ions of less than 150 ppm and the second reaction step took place at an excess amount of OH ions of more than 150 ppm, the second potassium lactate solution contained between 0.7 and 3.7 ppm magnesium ions. These Mg concentrations are much lower than the concentrations of 490 and 100 ppm magnesium ions obtained in example 6 of WO2005/052800.

Due to the low magnesium ion concentration, the second potassium lactate solutions obtained according to the processes of examples 3-5 are very suitable for application in electrodialysis for production of lactic acid.

TABLE 3

| Example | Mg in KL (ppm) |
|---|---|
| 1 | 2.1 |
| 2 | 82 |
| 3 | 3.7 |
| 4 | 2.1 |
| 5 | 0.7 |

Thus, an amount of excess OH ions in the first reaction step of less than 150 ppm and an excess amount of OH ions in the second reaction step of more than 150 ppm results in a second potassium lactate solution with a magnesium ion concentration of less than 50 ppm, while the process is more efficient because the Mg(OH)₂ formed in the first reaction step is separated more easily from the first potassium lactate solution.

1. A process for preparing an aqueous potassium lactate solution with a magnesium content of less than 50 ppm, the process comprising:
in a first reaction step, reacting solid-state magnesium lactate or an aqueous magnesium lactate suspension with KOH to form a first suspension of solid magnesium hydroxide in a first potassium lactate solution comprising magnesium ions, the reaction taking place under such conditions that the amount of excess OH ions in the first suspension is in the range of 20-150 ppm;
subjecting the first suspension to a solid-liquid separation step to separate solid magnesium hydroxide from the first potassium lactate solution;
in a second reaction step, subjecting the first potassium lactate solution resulting from the solid-liquid separation step to a further reaction with KOH to form a second suspension of solid magnesium hydroxide in a second potassium lactate solution, the reaction taking place under such conditions that the amount of excess OH ions in the second suspension is above 300 ppm; and
subjecting the second suspension to a solid-liquid separation step to separate solid magnesium hydroxide from the second potassium lactate solution, wherein the second potassium lactate solution has a magnesium ion content of less than 50 ppm.

2. The process according to claim 1, wherein the second potassium lactate solution has a magnesium ion content of less than 25 ppm.

3. The process according to claim 1, wherein the aqueous magnesium lactate suspension has a magnesium lactate content (calculated as magnesium lactate anhydrate) of at least 15 wt. % and at most 50 wt. % of the total weight of the suspension.

4. The process according to claim 1, wherein solid-state magnesium lactate comprises at least comprises at least 60 wt. %, magnesium lactate (calculated as magnesium lactate anhydrate) of the total weight of the solid.

5. The process according to claim 1, wherein in the first reaction step, the excess OH ions in the first suspension is in the range of 20-125 ppm.

6. The process according to claim 1, wherein in the second reaction step, the excess OH ions in the second suspension is above 400 ppm and at most 10000 ppm.

7. The process according to claim 1, wherein
the second reaction step is carried out at a temperature which is at least 20° C. higher than the temperature at which the first reaction step is carried.

8. The process according to claim 7, wherein the second reaction step is carried out at a temperature which is at least 25° C. higher than the temperature at which the first reaction step is carried out, and at most 75° C. higher than the temperature at which the first reaction step is carried out.

9. The process according to claim 1, wherein the first and the second reaction step are carried out at a temperature in the range of 20-100° C.

10. The process according to claim 1, wherein the temperatures in the first and second reaction steps are selected individually.

11. The process according to claim 1, wherein the pH in the second reaction step is at least 0.5 above the pH in the first reaction step.

12. The process according to claim 11, wherein the pH in the second reaction step is at least 0.7 above the pH in the first reaction step, and wherein the pH in the second reaction step is at most 2.5 above the pH in the first reaction step.

13. The process according to claim 1, further comprising providing the second potassium lactate solution to a reaction step in which potassium lactate is converted to lactic acid and KOH through water splitting electrodialysis.

14. A potassium lactate solution with a magnesium content of less than 50 ppm prepared by the process of claim 1.

15. A process for manufacturing lactic acid, the process comprising:
subjecting the potassium lactate solution of claim 14 to a reaction in which potassium lactate is converted to lactic acid and KOH through water splitting electrodialysis.

16. The process according to claim 13, wherein the KOH obtained after the electrodialysis is recycled to the first and/or second reaction step.

17. The process according to claim 16, wherein the potassium lactate solution with a magnesium content of less than 50 ppm is obtained by a process comprising:
fermenting a carbohydrate source to lactic acid in the presence of a microorganism, with the addition of magnesium hydroxide as neutralising agent, to form an aqueous fermentation medium comprising magnesium lactate,
optionally subjecting the aqueous medium to a biomass removal step,
optionally performing a concentration step to provide a magnesium lactate slurry,
optionally performing a liquid-solid separation step to obtain solid magnesium lactate,
optionally crystallising dissolved magnesium lactate to obtain solid magnesium lactate.

18. The process according to claim 16, wherein the process comprises recycling magnesium hydroxide obtained from subjecting the first and/or second potassium lactate suspension to solid-liquid separation to the step of fermenting a carbohydrate source.
